(19) United States
(12) Patent Application Publication
(10) Pub. No.: US 2021/0074432 A1
(43) Pub. Date: Mar. 11, 2021

(54) PREDICTIVE ANALYTICS FOR COMPLEX DISEASES

(71) Applicant: MEDSTAR HEALTH, INC., Columbia, MD (US)

(72) Inventor: David Z. Martin, Towson, MD (US)

(21) Appl. No.: 17/014,315

(22) Filed: Sep. 8, 2020

Related U.S. Application Data
(60) Provisional application No. 62/896,643, filed on Sep. 6, 2019.

(51) Int. Cl. G16H 50/30; G16H 50/20; G16H 10/60; G16H 20/00

(52) U.S. Cl. CPC G16H 50/30; G16H 20/00; G16H 10/60; G16H 50/20

(57) ABSTRACT

Systems and methods are provided for evaluating a patient with a complex medical condition. Data is extracted from one of a personal computing device of a patient, a social media platform, and a public record associated with the patient, and a set of metadata features are generated from the extracted data. A set of medical features are generated from an electronic health record of the patient. A clinical parameter is generated at a machine learning model based on the set of metadata features and the set of medical features. The clinical parameter represents one of a specific therapeutic intervention, a category of therapeutic interventions, a likelihood that a specific therapeutic intervention will be successful, and a likelihood that a patient will comply with a given therapeutic intervention.

### RELATED APPLICATIONS

This application claims priority from U.S. Provisional Application No. 62/896,643, filed 6 Sep. 2019 and entitled “PREDICTIVE ANALYTICS FOR COMPLEX DISEASES”, the subject matter of which is incorporated herein by reference in its entirety.

### TECHNICAL FIELD

This invention relates to medical diagnostic systems, and more particularly, to predictive analytics for complex diseases.

### BACKGROUND

A clinical decision support system provides an interactive knowledgebase system to assist physicians and other health professionals in various health care related tasks. One purpose of such systems is to assist health care providers at the point of care. For example, some systems can help determine diagnoses, analyze various patient information as well as to monitor conditions of on-going procedures. Clinic decision support encompasses a variety of tools to enhance decision-making in the clinical workflow. These tools include computerized alerts and reminders to care providers and patients; clinical guidelines; condition-specific order sets; focused patient data reports and summaries; documentation templates; diagnostic support, and contextually relevant reference information, among other tools.

### SUMMARY

In accordance with one aspect of the invention, a method is provided. Data is extracted from one of a personal computing device of a patient, a social media platform, and a public record associated with the patient, and a set of metadata features are generated from the extracted data. A set of medical features are generated from an electronic health record of the patient. A clinical parameter is generated at a machine learning model based on the set of metadata features and the set of medical features. The clinical parameter represents one of a specific therapeutic intervention, a category of therapeutic interventions, a likelihood that a specific therapeutic intervention will be successful, and a likelihood that a patient will comply with a given therapeutic intervention.

In accordance with another aspect of the present invention, a system is provided. The system includes a processor and a non-transitory computer readable medium storing executable instructions for generating a clinical parameter for a patient. A network interface receives a first set of data, from one of a personal computing device of a patient, a social media platform, and a public record associated with the patient, and a second set of data from an electronic health record of the patient. A feature extractor generates a set of metadata features and a set of demographic features from the first set of data and a set of medical features from the second set of data. A machine learning model generates a clinical parameter based on the set of metadata features, the set of demographic features, and the set of medical features. The clinical parameter represents one of a specific therapeutic intervention, a category of therapeutic interventions, a likelihood that a specific therapeutic intervention will be successful, and a likelihood that a patient will comply with a given therapeutic intervention.

In accordance with yet another aspect of the invention, a method is provided. Data is extracted from one of public records, Internet searches, existing medical records, and the patient's self-reporting, and a set of demographic features are generated from the extracted data. A set of medical features are generated from an electronic health record of the patient. A clinical parameter is generated at a machine learning model based on the set of demographic features and the set of medical features. The clinical parameter represents one of a specific therapeutic intervention, a category of therapeutic interventions, a likelihood that a specific therapeutic intervention will be successful, and a likelihood that a patient will comply with a given therapeutic intervention

### BRIEF DESCRIPTION OF THE DRAWINGS

FIG. 1 illustrates a diagnostic system for evaluating a patient with a complex medical condition in accordance with an aspect of the present invention;

FIG. 2 illustrates an example of a system for assisting a medical practitioner in determining a course of treatment for a wound to a patient in accordance with an aspect of the present invention;

FIG. 3 illustrates a method for evaluating a patient with a complex medical condition in accordance with an aspect of the present invention;

FIG. 4 illustrates another method for evaluating a patient with a complex medical condition in accordance with an aspect of the present invention; and

FIG. 5 is a schematic block diagram illustrating an exemplary system of hardware components capable of implementing examples of the systems and methods disclosed herein.

### DETAILED DESCRIPTION

A “metadata feature,” as used herein, is a feature derived from activity by a patient outside of a medical environment that is relevant to the patient's mental and physical condition. Metadata features can include commercial transactions, Internet usage, social media postings, telephone communications, and SMS communication. Metadata can be derived from public records, Internet searches, social media platforms, and an application installed on personal computing device used by the patient. Where the metadata features are derived from data extracted from the application, it will be appreciated that the data can represent both activity on the personal computing device prior to onset of symptoms associated with a given disease or disorder as well as activity after the onset of symptoms.

A “demographic feature,” as used herein, refers to characteristics of a patient that are not linked to a physical or mental characteristic or status of a patient that are relevant to the patient benefiting from on a prescribed course of care after treatment. Examples of demographic features can include the patient's level of education, marital status, insurance status, area of residence, employment status, and residence status.

A “personal computing device,” as used herein, is a computing device used by the patient that can include any of a mobile device (e.g., a mobile phone or tablet), a laptop, a desktop computer, a smart television, or a streaming media device.

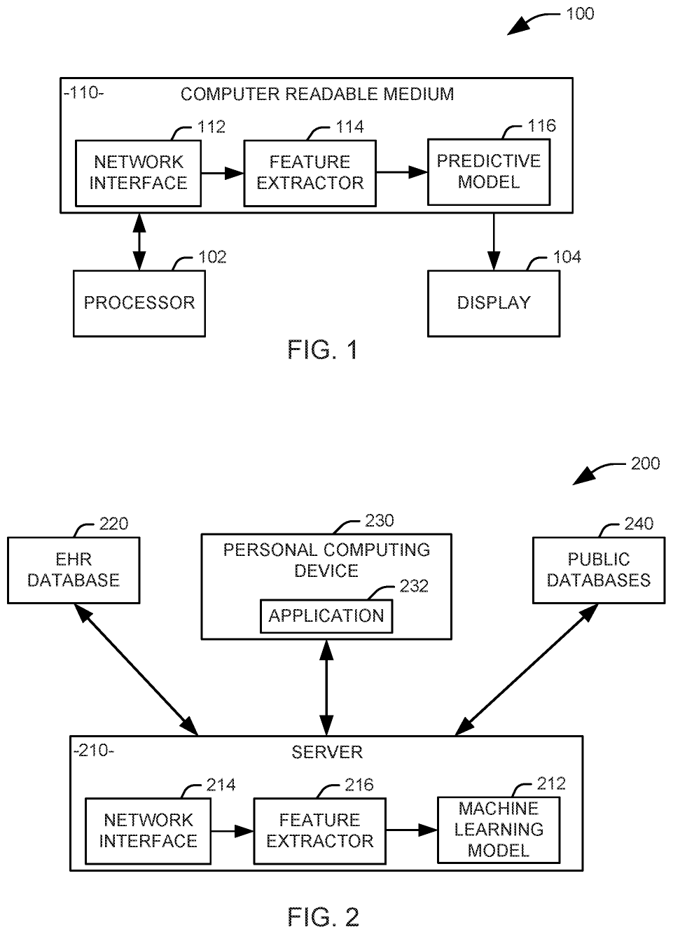

FIG. 1 illustrates a diagnostic system 100 for evaluating a patient with a complex medical condition in accordance with an aspect of the present invention. The diagnostic system 100 includes a processor 102, a display 104, and a non-transitory computer readable medium 110 storing computer readable instructions, executed by the processor 102. The executable instructions stored on the non-transitory computer readable medium 110 include a network interface 112 through which the system 100 communicates with other systems (not shown) via a network connection, for example, an Internet connection and/or a connection to an internal network. In the illustrated example, the other systems can include an electronic health records (EHR) system that stores medical information for the patient, social media platforms, search engines, public record databases, and an application installed on personal computing device used by the patient.

Information retrieved via the network interface 112 is provided to a feature extractor 114 that extracts a plurality of features for use at a predictive model 116. It will be appreciated that many of the sources accessed via the network interface 112 can be in the form of unstructured and semi-structured text, and the feature extractor 114 can include natural language processing algorithms for extracting data from unstructured text as well as appropriate templates and queries for extracting specific fields from semi-structured data sources.

In one example, dealing with wound care, a number of features can be extracted from an EHR system or other medical records representing the physical condition of the patient, characteristics of the wound itself, and laboratory testing. While these factors are all relevant to the patient's physical condition, the inventors note that, particularly in wound care, factors beyond the physical condition of the patient can be relevant to the expected recovery and influence the intervention selected for the patient. Accordingly, the feature extractor 114 can also extract demographic features from publically available databases, including public records, social media platforms, and Internet searches. Metadata parameters can be extracted from public records, Internet searches, and social media platforms. In one example, many of these metadata parameters, as well as additional features, can be provided by an application installed on a personal computing device used by the patient.

A predictive model 116 uses the plurality of extracted features to assign a clinical parameter to the patient. The clinical parameter can be a continuous or categorical parameter representing a patient's response to treatment. For example, a continuous clinical parameter can represent a likelihood that the patient would respond to a given intervention, a degree of improvement expected for the patient given a specific intervention, and a likelihood that a patient will comply with a given therapeutic intervention. A categorical clinical parameter can represent ranges of the quantities listed, specific interventions, and categories of interventions. The clinical parameter can then be provided to the user at the display 104.

FIG. 2 illustrates another example of a system 200 for assisting a medical practitioner in determining a course of treatment for a wound to a patient in accordance with an aspect of the present invention. The system 200 includes a server 210 storing machine executable instructions for retrieving patient data from various sources 220, 230, and 240, and classifying the patient into one of a plurality of classes at a machine learning model 212 using the patient data. To this end, the server 210 stores a network interface 214 that interacts with each of the data sources 220, 230, and 240 to obtain the patient data.

For example, the network interface 214 can retrieve patient data from an electronic health records (EHR) database 220. The patient data can be provided to a feature extractor 216 that extracts classification features from the retrieved patient data relevant to the patient's physical condition and the condition of the wound. One set of features can be extracted as categorical parameters describing the patient generally, including past medical history, expressed for example, as a set of parameters representing existing conditions and previous procedures, known allergies, medications currently taken by the patient, prior and scheduled medical appointments, the patient's age, and the patient's sex. Another set of parameters can represent the wound and the affected portion of the body itself and can include parameters describing the type, dimensions, and age of the wound, the status of the periwound tissue, wound drainage, wound bed quality, edema, erythema, any pallor in the affected region, warmth or coolness in the wound or proximate tissue, the healing trajectory of the wound, and, when the wound in on a limb, the status of the contralateral limb. Another set of features can represent a current health of the patient, including the results of cardiovascular and pulmonary exams, vital signs, reported pain, and ambulatory status. It will be appreciated that each of these parameters can be extracted from appropriate fields in a medical record or determined via natural language processing of free text.

Still another set of parameters can be extracted from laboratory data stored in the EHR 220. It will be appreciated that these parameters are generally numerical data and can be extracted from known fields within the report. Examples include random blood glucose readings, HgbA1C values, creatinine, albumin, C-reactive protein, and prealbumin levels, results from liver function tests and complete blood count tests, prothrombin times, and erythrocyte sedimentation rates. Parameters can also be extracted from automated analysis of medical images or technician reports for chest X-rays, echocardiograms, arterial doppler scans, computer tomography images, ultrasound images, magnetic resonance images, positron emission tomography, and other medical imaging and nuclear medicine techniques.

The network interface 214 can also retrieve information from an application 232 installed on a personal computing device 230 used by the patient. For example, the application can monitor social media use to determine the frequency and content of the patient's social media postings. The patient's search history and visited websites can be monitored in a similar fashion. The content of the searches and social media postings can be evaluated for key words that could be indicative of the patient's status, for example, words representing symptoms associated with complications with the wound. The frequency of the patient's postings and Internet usage can be indicative of the mental and physical state of the patient. Where the personal computing device is a cellular phone, a call log and text history of the patient can also be evaluated for the frequency of contact with others, with texts also evaluated for key words relevant to the wound. The application can also interact with a location service on the phone as well as sensors or application tied in with fitness devices to gauge the patient's activity. Finally, the application can monitor the battery charge of the device, as patients who are reliable in maintaining the charge in the device's battery may be expected to be reliable in following a course of care. The information provided from the application 232 can be provided to the feature extractor 216 to generate at least a portion of a set of metadata features based on the information from the application.

The network interface 214 can also retrieve information from various public databases 240, including public records, social media platforms, and Internet searches. Demographic features can be extracted from this information at the feature extractor 216, as well as the patient's medical records from the EHR 220 and self-reported information from the patient. In one example, the patient can provide the self-reported data through the application 232, although it can also be provided during an encounter with a medical professional for treatment of the wound or as a submitted paper or electronic form. Demographic features can include the patient's level of education, marital status, insurance status, area of residence (as determined, for example, by the patient's zip code and/or area code), employment status, and residence status (e.g., apartment, ranch home, multi-story home, nursing home, trailer, etc.). Each of these factors can be indicative of the patient's likelihood of following through on a course of care after a particular intervention, the resources available to the patient to pursue any necessary follow-up care, and the unique challenges (e.g., stairs and home upkeep) that might be present for the patient. To use a crude example, a patient living in a multistory home in a zip code known to experience harsh winters is likely to present a significantly higher fall risk than a patient in a single story home in a warmer climate during the winter months. It will be appreciated, however, that the machine learning model 212, once trained, would be expected to find more subtle correlations between the patient's metadata parameters and the optimal intervention for the patient.

Metadata features can also be extracted from data retrieved from publicly available databases, including court records, credit ratings, and general background checks. Internet searches can locate application profiles and mentions of the patient in media. The patient can also opt to share bank records, credit data, and overall transaction data for a given period. Features extracted from this information can be used as an indication both of potential stress on the patient as well as the availably of resources to support a course of care. Social media posts by the client can be retrieved to determine a frequency and content of the patient's postings. These features can be added to the metadata features extracted from data received at the application 232 to provide additional insight into the patient's activity outside of the medical environment.

The extracted features are provided to the machine learning model 212 to assign the patient to one of a plurality of classes representing potential interventions that can be prescribed to the patient. A given class can represent a likelihood that the patient's wound will respond to a given treatment or general class of treatments or a recommendation for a specific intervention, category of interventions, referral, or additional testing. In one implementation, the classification is tied to a specific intervention and is binary, with “likely to respond” and “unlikely to respond” classes, although it will be appreciated that additional classes could be included, for example, classes representing ranges of likelihoods that the patient will respond to treatment. In another implementation, the classes could represent a patient's expected response to the treatment, for example, “improving”, “no change”, and “degrading” classes, or classes representing expected degrees of response to treatment. And, of course, the classes can each be a recommended next action for the patient, including interventions, referrals, and additional tests.

Additional tests that might be recommended by the system 200 include laboratory tests, such as testing for various levels of substances in blood (e.g., blood glucose, HgbA1C, creatinine, albumin, C-reactive protein, and prealbumin), liver function tests, complete blood counts, prothrombin time tests, blood typing and screening, and erythrocyte sedimentation rate testing, as well as diagnostic testing, such as X-rays, computed tomography, magnetic resonance imaging, echocardiograms, electrocardiographs, and vascular testing. Referrals can be to hospitals, inpatient or outpatient rehabilitation, home health care, a medical home, hospice, an incentive program, or consults with vascular specialist, a cardiologist, an endocrinologist, an internal medicine specialist, or an infectious disease specialist. Possible interventions include diabetes education, a nutrition plan, hemodialysis, diuretics, systemic or topical antibiotics, wound bed or excisional debridement, compression, elevation, advanced modality evaluation, synthetic or natural acellular skin substitutes, synthetic or natural cellular skin substitutes, topical, nasal, or hyperbaric oxygen, skin graft, flap reconstruction, amputation, tendon rebalancing, orthopedic reconstruction, offloading shoes or boots. frequent turning and repositioning, a specialty bed, mattress, or cushion, urinary or fecal diversion, compression therapy, lymphedema pumps, total contact casts, negative pressure therapy, growth factors, biopsy, hydrogel, alginate, collagen, silver, steroids, non-adherent dressings, and various petroleum products. It will be appreciated that, while any or all of these tests, referrals, and interventions can represent a class or set of classes in the machine learning model 212, they are not all necessary for a system 200 in accordance with the present invention, and they do not provide an exhaustive list of potential classes even in the example implementation of the system for wound care.

The machine learning model 212 can utilize one or more pattern recognition algorithms, each of which analyze the extracted features or a subset of the extracted features to classify the patients into one of the plurality of classes and provide this information to a medical professional at an appropriate output device (not shown) such as a display. Where multiple classification or regression models are used, an arbitration element can be utilized to provide a coherent result from the plurality of models. Each model is trained on training data representing various classes of interest. The training process of a given classifier will vary with its implementation, but training generally involves a statistical aggregation of training data into one or more parameters associated with the output class. Any of a variety of techniques can be utilized for the classification algorithm, including support vector machines, regression models, self-organized maps, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used. In the illustrated implementation, the classifier includes one or both of a support vector machine and a random forest classifier. While the illustrated implementation utilizes one or more classifiers to categorize the patient, it will be appreciated that a regression model or similar approach can be employed to give a continuous, as opposed to a categorical output, for example, representing a likelihood that a patient will respond to a given treatment.

Where the mobile device is used, additional data on the patient can be collected after any therapeutic intervention to access the efficacy of the intervention given. In particular, an activity level of the patient, as determined by kinematic sensors on a mobile device or GPS data, can be compared to previously captured data to determine if the patient's level of activity has changed. The content of Internet searches, social media posts, and similar content can also be monitored for keywords indicative of the patient's mental and physical state. In one implementation, this data can be provided to a physician for review. Alternatively or additionally, the information can be used as feedback to the machine learning model to refine the parameters defining the model. For example, the additional data can be compared to the previous data to assign the patient to one of the plurality of classes, and the patient's data can be utilized as training data in a retraining of the model.

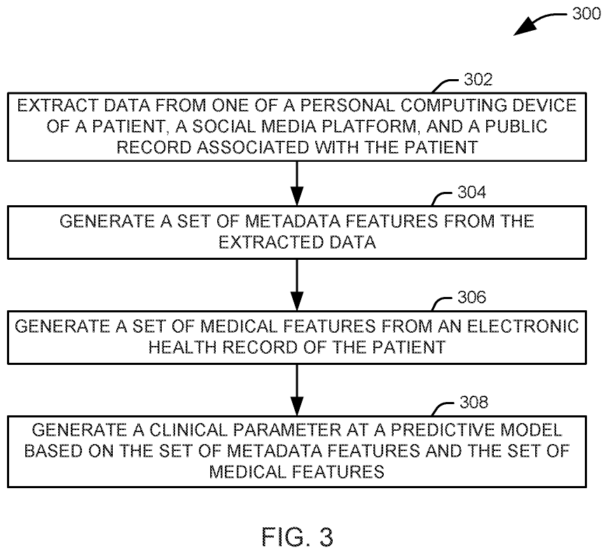

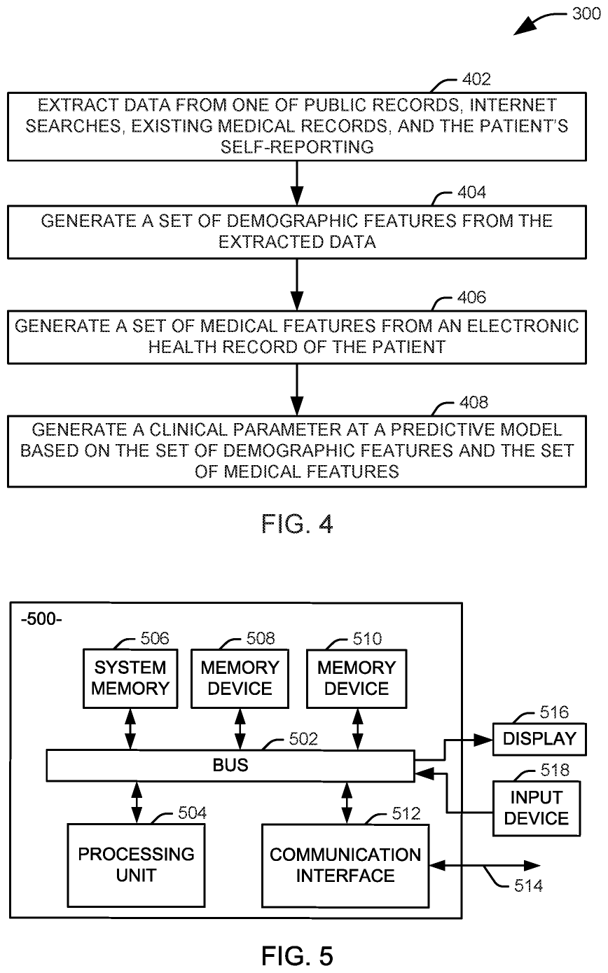

In view of the foregoing structural and functional features described above, example methods will be better appreciated with reference to FIGS. 3 and 4. While, for purposes of simplicity of explanation, the example methods of FIGS. 3 and 4 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 3 illustrates a method 300 for evaluating a patient with a complex medical condition in accordance with an aspect of the present invention. At 302, data is extracted from one of a personal computing device of a patient, a social media platform, and a public record associated with the patient. In one example, an application stored on the personal computing device can be used to monitor online activity of the patient. At 304, a set of metadata features can be generated from the extracted data. For example, metadata parameters can include a representative level of battery charge in the personal computing device of the patient, a frequency of occurrence of a set of key words in Internet searches performed by the patient on the personal computing device, a parameter representing a frequency of Internet usage by the patient on the personal computing device, and a parameter representing a frequency of social media posting by the patient.

At 306, a set of medical features are generated from an electronic health record of the patient. The medical features can include, for example, biometric parameters, medical history, and other continuous and categorical parameters representing the physical and mental health of the patient. In one example, in addition to the metadata features and the medical features, a set of demographic features for the patient can also be extracted from one of public records, Internet searches, existing medical records, and the patient's self-reporting. Examples of demographic parameters include one of an area code, a zip code, and a city associated with a residence of the patient, a categorical parameter representing a residential status of the patient, and a parameter representing a level of education of the patient.

At 308, a clinical parameter is generated at a predictive model based on the set of metadata features and the set of medical features. The clinical parameter can represent, for example, one of a specific therapeutic intervention, a category of therapeutic interventions, a likelihood that a specific therapeutic intervention will be successful, and a likelihood that a patient will comply with a given therapeutic intervention. Where demographic features are extracted, the clinical parameter can be generated from the set of metadata features, the set of demographic features, and the set of medical features. In one example, the therapeutic interventions can represent various interventions that can be applied for a wound of the patient, and the extracted parameters can be selected to relate to characteristics of the wound, characteristics of the patient, and the patient's activity outside of a medical setting. Where the clinical parameter either represents a selected therapeutic intervention or indicates that an intervention is likely to be successful, the therapeutic intervention can be provided to the patient.

FIG. 4 illustrates another method 400 for evaluating a patient with a complex medical condition in accordance with an aspect of the present invention. At 402, data is extracted from one of public records, Internet searches, existing medical records, and the patient's self-reporting. At 404, a set of demographic features can be generated from the extracted data. For example, demographic parameters can include one of an area code, a zip code, and a city associated with a residence of the patient, a categorical parameter representing a residential status of the patient, and a parameter representing a level of education of the patient. At 406, a set of medical features are generated from an electronic health record of the patient. The medical features can include, for example, biometric parameters, medical history, and other continuous and categorical parameters representing the physical and mental health of the patient.

At 408, a clinical parameter is generated at a predictive model based on the set of demographic features and the set of medical features. The clinical parameter can represent, for example, one of a specific therapeutic intervention, a category of therapeutic interventions, a likelihood that a specific therapeutic intervention will be successful, and a likelihood that a patient will comply with a given therapeutic intervention. In one example, the therapeutic interventions can represent various interventions that can be applied for a wound of the patient, and the extracted parameters can be selected to relate to characteristics of the wound, characteristics of the patient, and the patient's activity outside of a medical setting. Where the clinical parameter either represents a selected therapeutic intervention or indicates that an intervention is likely to be successful, the therapeutic intervention can be provided to the patient.

FIG. 5 is a schematic block diagram illustrating an exemplary system 500 of hardware components capable of implementing examples of the systems and methods disclosed herein. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings.

Additionally or alternatively, the system 500 can access an external data source or query source through the communication interface 512, which can communicate with the system bus 502 and the communication link 514.

In operation, the system 500 can be used to implement one or more parts of a system for performing predictive analytics for complex diseases in accordance with the present invention. Computer executable logic for performing the predictive analytics resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A method comprising:
extracting data, from one of a personal computing device of a patient, a social media platform, and a public record associated with the patient;
generating a set of metadata features from the extracted data;
generating a set of medical features from an electronic health record of the patient; and
generating a clinical parameter at a machine learning model based on the set of metadata features and the set of medical features, the clinical parameter representing one of a specific therapeutic intervention, a category of therapeutic interventions, a likelihood that a specific therapeutic intervention will be successful, and a likelihood that a patient will comply with a given therapeutic intervention.

2. The method of claim 1, further comprising generating a set of demographic features for the patient from one of public records, Internet searches, existing medical records, and the patient's self-reporting, wherein generating the clinical parameter comprises generating the clinical parameter based on the set of metadata features, the set of demographic features, and the set of medical features.

3. The method of claim 2, wherein the set of demographic parameters includes one of an area code, a zip code, and a city associated with a residence of the patient.

4. The method of claim 2, wherein the set of demographic features comprises a categorical parameter representing a residential status of the patient.

5. The method of claim 2, wherein the set of demographic features include a level of education of the patient.

6. The method of claim 1, wherein the set of metadata parameters include a representative level of battery charge in the personal computing device of the patient.

7. The method of claim 1, wherein the set of metadata parameters comprises at least one parameter representing a frequency of occurrence of a set of key words in Internet searches performed by the patient on the personal computing device.

8. The method of claim 1, wherein the set of metadata parameters comprises at least one parameter representing a frequency of Internet usage by the patient on the personal computing device.

9. The method of claim 1, the set of metadata parameters comprises at least one parameter representing a frequency of social media posting by the patient.

10. The method of claim 1, wherein the clinical parameter is a categorical parameter representing a selected therapeutic intervention, the method further comprising providing the selected therapeutic intervention to the patient.

11. The method of claim 1, the clinical parameter representing one of a specific therapeutic intervention for treating a wound, a category of therapeutic interventions for treating the wound, a likelihood that a specific therapeutic intervention for treating the wound will be successful, and a likelihood that a patient will comply with a given therapeutic intervention for treating the wound.

12. A system comprising:
a processor; and
a non-transitory computer readable medium storing executable instructions for generating a clinical parameter for a patient, the executable instructions comprising:
a network interface that receives a first set of data, from one of a personal computing device of a patient, a social media platform, and a public record associated with the patient and a second set of data from an electronic health record of the patient;
a feature extractor that generates a set of metadata features and a set of demographic features from the first set of data and a set of medical features from the second set of data; and
a machine learning model that generates a clinical parameter based on the set of metadata features, the set of demographic features, and the set of medical features, the clinical parameter representing one of a specific therapeutic intervention, a category of therapeutic interventions, a likelihood that a specific therapeutic intervention will be successful, and a likelihood that a patient will comply with a given therapeutic intervention.

13. The system of claim 12, wherein the set of demographic features includes one of a categorical parameter representing a residential status of the patient, a parameter representing a level of education of the patient, an area code associated with a residence of the patient, a zip code associated with the residence of the patient, and a city associated with the residence of the patient.

14. The system of claim 12, wherein the set of metadata parameters include a representative level of battery charge in the personal computing device of the patient, a frequency of occurrence of a set of key words in Internet searches performed by the patient on the personal computing device, a parameter representing a frequency of Internet usage by the patient on the personal computing device, and a parameter representing a frequency of social media posting by the patient.

15. The system of claim 12, further comprising an application stored on the personal computing device of the patient that monitors activity of the patient on the personal computing device, the set of metadata features including at least one feature representing the monitored activity.

16. A method comprising:
extracting data, from one of public records, Internet searches, existing medical records, and the patient's self-reporting;
generating a set of demographic features from the extracted data;
generating a set of medical features from an electronic health record of the patient; and
generating a clinical parameter at a machine learning model based on the set of demographic features and the set of medical features, the clinical parameter representing one of a specific therapeutic intervention, a category of therapeutic interventions, a likelihood that a specific therapeutic intervention will be successful, and a likelihood that a patient will comply with a given therapeutic intervention.

17. The method of claim 16, further comprising generating a set of metadata features for the patient from one of a personal computing device of a patient, a social media platform, and a public record associated with the patient, wherein generating the clinical parameter comprises generating the clinical parameter based on the set of metadata features, the set of demographic features, and the set of medical features.

18. The method of claim 16, wherein the set of demographic parameters includes one of an area code, a zip code, and a city associated with a residence of the patient.

19. The method of claim 16, wherein the set of demographic features comprises a categorical parameter representing a residential status of the patient.

20. The method of claim 16, wherein the set of demographic features include a level of education of the patient.
